(19) United States
(12) Patent Application Publication
(10) Pub. No.: US 2022/0409445 A1
(43) Pub. Date: Dec. 29, 2022

(54) DISPOSABLE COMPOSTABLE DIAPER AND FULFILLMENT SERVICE

(71) Applicant: Sergio Radovcic, Phoenix, AZ (US)

(72) Inventor: Sergio Radovcic, Phoenix, AZ (US)

(21) Appl. No.: 17/901,652

(22) Filed: Sep. 1, 2022

Related U.S. Application Data
(62) Division of application No. 16/520,259, filed on Jul. 23, 2019.

(51) Int. Cl. A61F 13/49; A61F 13/494; A61F 13/15; A61F 13/42

(52) U.S. Cl. CPC A61F 13/49007; A61F 13/49413; A61F 13/15252; A61F 13/42; A61F 2013/530007

(57) ABSTRACT

A disposable diaper having a first sheet of viscose fiber material disposed against a child's skin, an acquisition and diffusion layer (ADL) interposed over top of the first sheet of viscose fiber, a coating layer of tissue fiber interposed over top of the ADL layer, an absorbency core layer interposed over top of the coating layer, a permeable layer of polymer film interposed over top of the absorbency core layer, and a second sheet of viscose fiber material overlaid over top of the permeable polymer film layer, the second sheet functioning as the exterior sheet of the disposable diaper.

### CROSS-REFERENCE TO RELATED DOCUMENTS

The present US non-provisional patent application claims priority, as a divisional patent application, to earlier filed co-pending U.S. patent application Ser. No. 16/520,259 filed on Jul. 23, 2019.

### BACKGROUND OF THE INVENTION

### 1. Field of the Invention

The present invention is in the field of postnatal support and fulfillment services and pertains particularly to a unique organic diaper and methods and apparatus for providing essential post natal products including organic diapers to clients of a digital tracking and recommendation service.

### 2. Discussion of the State of the Art

In the field of physical fitness, maintaining physical health and fitness is a concerted passion for people all over the world. Aside from user-operated fitness machines, there are many consumer products that have been developed to help people track their activities and pursue goals related to fitness. These products include mobile and often wearable electronics devices for tracking activity and certain states of a user performing the tracked activity at the time. An activity that may be tracked might be running, walking, swimming, dieting, lifting, cycling, and other activities pursued with a goal of attaining a level of physical fitness for the tracked user. It is estimated that over two million users are currently tracking their activities and pursuing some fitness goal.

Modular electronics devices are known to be able to track distance and time, running or walking, monitor heart rates, and blood pressures, sweat rates, breathing rates, and can calculate simple results such as the number of calories burned, electrolyte loss and so on during the activity. Mobile phone applications have been created that enable a user being tracked to upload data to a remote server where the data may be processed, and recommendations may be later sent to the user being tracked. Some of these products include but are not limited to running watchers, mobile phone apps, such as RunKeeper or Strava, or like activity trackers, such as FitEit or Jawbone. Forums are also a popular way of tracking physical fitness and health. These also allow the user to receive detailed feedback and recommendations. More particularly, these products work individually and do not aggregate the data from disparate data sources to produce personalized recommendations.

The inventor is aware of a network service model for tracking physical activity for a client that is pregnant with a child or is past pregnancy and raising one or more small children within a 720 day period after pregnancy. The service tracks client activities and then consults with available client provided data and third party data to make important nutritional and product recommendations to the client. The system encompasses a network-hosted service model that includes at least one network connected server(s) and associated data repositories controlled by a service provider entity.

The client is provided with at least one dedicated activity tracking device including a wearable electronic pendant including a sensor tracking at least motion, time of tracked motion, and direction and distance of tracked motion. Another available device to the client tracks the amount of liquids a client consumes while engaging in activities. Software is provided to the client to install on a mobile phone so the client may use the device to interact with the service and to authorize transactions resulting from any recommendations made by the service. Software is also provided at a network-hosted server to perform calculations based on client data and third party data that are the basis for making recommendations to the client. The client may get recommendations supported by processing activity data, first and third party available data and knowledge data contributed to a knowledge base maintained for the purpose of providing searchable data about prenatal period, the trimesters of pregnancy, and post natal care of a mother and child.

The fulfillment part of the system functions to host transactions for products available to the client through the service and to ensure that those products are delivered to the client. The service model is adapted to determine and address at least the nutritional needs of the client through a pregnancy of approximately 280 days and during a post pregnancy period of approximately 720 days. The service may supply vitamins, protein supplements, electrolytes, organic diapers, organic baby wipes, organic lotions, organic powders, and baby foods or formulas.

Disposable organic diapers are included in the products that may be ordered and shipped to clients of the service based on orders placed by the client's. Disposable diapers began replacing cloth diapers in the mid 1950s. The reason for disposable diapers was to provide a product without requiring work to wash and reuse the product. Cloth diapers cost an average of $2,400.00 per child whereas disposable diapers cost less, around $1500.00. A problem with disposable diapers is that they contain many inorganic materials like plastic, super absorbent poly gels and wood fluff materials contaminated with heavy metals such as Tributyl-tin. Chemicals are sometimes added to mask odors. Added chemicals may include chlorine, various dyes, fragrances, phthalates (plastic softeners) and more. Another problem with non-organic disposable diapers is they may take up to 500 years to compost.

More recently, diaper manufactures have developed more organic versions of disposable diapers that are more compostable (decompose faster) and reduce the number of non-organic materials that are typically used in their manufacture. However, inorganic materials are still used such as the super absorbent chemical sodium polyacrylate or sodium polycarbonate and some synthetic materials such as plastic softened with a plastic softener (plasticizers). At the time of this writing, the best organic disposable diaper currently available takes up to 6 months to decompose in sunlight and oxygen.

Another challenge to using organic or green disposable diapers is that they still may end up in a land fill where they may not decompose as advertised because of being buried in the land fill so that sunlight and oxygen required for decomposition is not present. Still other challenges exist in providing a true organic decomposable disposable diaper in an affordable and efficient fulfillment process. It may also be desirable to utilize a composting service to decompose used organic diapers as opposed to burying them in a land fill where decomposition is unlikely or impossible within a reasonable time.

Therefore, what is clearly needed is a decomposable disposable organic diaper product that eliminates or reduces the problems cited above with current disposable diapers and a fulfillment service that may account for diaper use, fulfill emergency requirements, and disposal, and that may automatically reorder an amount consistent with actual diaper use statistics.

### BRIEF SUMMARY OF THE INVENTION

A disposable diaper is provided and includes a first sheet of viscose fiber material disposed against a child's skin, an acquisition and diffusion layer (ADL) interposed over top of the first sheet of viscose fiber, a coating layer of tissue fiber interposed over top of the ADL layer, an absorbency core layer interposed over top of the coating layer, a permeable layer of polymer film interposed over top of the absorbency core layer, and a second sheet of viscose fiber material overlaid over top of the permeable polymer film layer, the second sheet functioning as the exterior sheet of the disposable diaper.

In one embodiment, the absorbency core layer contains a PH strip that changes color to indicate level of acidity within the core layer, the color indicated visible to a user and matchable to a chart listing PH levels associated with the colors. In a preferred embodiment, the first and second sheets of viscose fiber are compostable bamboo fiber. In this embodiment, the absorbency core layer further includes a super absorbent polymer (SAP) derived from biodegradable plant cellulose and a chlorine free compostable wood pulp material. In a variation of this embodiment, the SAP is created by inverse suspension polymerization to avoid chemicals used in emulsification processes.

In one embodiment, the first and second sheets are micro perforated to enforce dryness against the child's skin and at the external side of the diaper. In a preferred embodiment, the disposable diaper is biodegradable and compostable as a direct result of selective material incorporation and selective material exclusion. In one embodiment, the permeable layer of poly film is biodegradable plant based polymer that contains enzymes that enhance biodegrading efficiency of the disposable diaper.

According to another embodiment of the present invention, a network-hosted order and fulfillment service for biodegradable diapers is provided and includes a first non-transitory medium coupled to a first network server having connection to the network, the first server coupled to a data repository the non-transitory medium containing thereon instruction causing the server to (a) provide for download and install a network browser-based application to a user's mobile communications device having network connect capability, (b) via the application as a user interface, receive a request from the user for a subscription to the fulfillment service, (c) via the application as a user interface, establish a service account for a user upon request, (d) via the application as a user interface, collect all of the data required to redirect the order request to a second network server functioning as a secure transaction server, and (e) via the application as a user interface, sync data between the first server and the mobile communications device upon established connection, the data synced displayable through at least one electronic page of the browser-based application on the user's mobile device.

The network-hosted order and fulfillment service for biodegradable diapers further comprises a second non-transitory medium coupled to a third network server having connection to the network, the third server coupled to a data repository and having network connection to the first server, and to the second server, the second non-transitory medium containing thereon instruction causing the third server to, (a) receive a notification to track a job order from a warehouse distribution hub, (b) assign the job order to active tracking state and confirm the active tracking state including sending any resulting underlying tracked data back to the first network server, (c) receive one or more activity updates from the distribution hub regarding at least one updated status of the tracked order, (d) forward the data from step (c), to the first server for storage into the data repository coupled to the first server, (e) receive from the first server or from a tracking device having network connect and report capability use statistics relative to diaper use and diaper disposal, (f) pass the data to a fourth server coupled to a data repository and having connection to the third server, the second server, and the first server, a third non-transitory medium coupled to the fourth server containing thereon instruction causing the fourth server to, (g) analyze the received data of step (f), compare the data results with current and or future shipment numbers expected each delivery, and (h) make a determination whether or not to change order volume of the tracked order with respect to adjusting a shipping quantity to align more closely with the analyzed disposal statistics.

In one embodiment, the fourth server makes recommendations based on analysis of data received from the third server. In one embodiment, the third and fourth servers are dedicated server applications hosted on a single network-connected machine.

### BRIEF DESCRIPTION OF THE SEVERAL VIEWS OF THE DRAWINGS

FIG. 1 is an overhead view of the back side of an organic disposable diaper according to an embodiment of the present invention.

FIG. 2 is an underside view of the diaper of FIG. 1.

FIG. 3 is a block diagram depicting the basic material layers of the diaper of FIG. 1.

FIG. 4 is a side elevation view of a diaper pail repository for used diapers depicted in a closed and locked position.

FIG. 5 is a side elevation view of the diaper pail of FIG. 4 depicted unlocked and opened to receive a used diaper.

FIG. 6 is an architectural overview of a communications network supporting a diaper fulfillment service according to an embodiment of the present invention.

FIG. 7 is a process flow chart depicting process steps for starting and managing a diaper order according to an embodiment of the present invention.

FIG. 8 is a process flow chart depicting process steps for operating a diaper repository to accept diapers for disposal and updating use statistics according to an embodiment of the present invention.

FIG. 9 is an elevation view of exemplary screen shots 900 of the mobile application of FIG. 6.

FIG. 10 is an elevation view of exemplary screen shots 1000 of the mobile application of FIG. 6.

FIG. 11 is a sequence diagram depicting interaction between network nodes engaged in the service of the present invention.

FIG. 12 is a block diagram depicting electronics components of the diaper pail of FIG. 4 according to one embodiment of the invention.

FIG. 13 is a process flow chart depicting steps for instating and full filling an emergency shipment of diapers.

### DETAILED DESCRIPTION OF THE INVENTION

The inventor provides a unique biodegradable disposable diaper that is compostable and a network enabled process for obtaining timely access to them and for disposing of them in a manner which is completely accountable. The present invention is described in enabling detail using the following examples, which may describe more than one relevant embodiment falling within the scope of the present invention.

It is a goal of the present invention to provide a biodegradable diaper that is soft, absorbent, and contains little or no materials that are synthetic and therefore not compostable in a shorter period of time compared with current products available. It is a further goal of the present invention that a diaper be provided that functions organically to absorb urine and fecal matter and may also indicate via a visible sensor or sensors to a user that the diaper has become soiled and requires changing Another goal of the present invention is to make such diapers available to users in a manner that does not impose adversity on the user in the form of any insecurity over availability including in emergencies or when the user is mobile. A further goal of the invention is to enable an automated accounting of the acquisition and use of diapers and to enable automated notification to a referred or user-selected composting service when there are enough disposed diapers collected for a pick up and transfer to a composting facility.

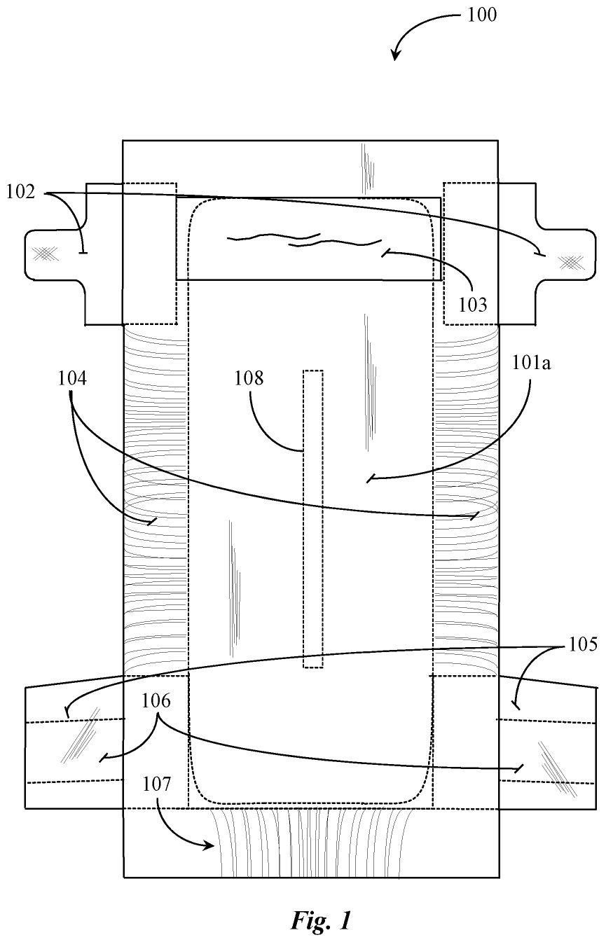

FIG. 1 is an overhead view of the back exterior side of an organic disposable diaper 100 according to an embodiment of the present invention. The term back side is meant to be analogous with the external visible parts and surfaces of the organic diaper relative to materials and functional components thereof when the diaper is worn. Diaper 100 is constructed from a variety of organic biodegradable materials where such materials and their arrangements in the construction of the diaper are critical factors in improving the compost-ability of the diaper compared with current disposable diapers that are available without sacrificing absorbent function and comfort.

The top sheet or back sheet of diaper 101a is fabricated from organically processed bamboo described succinctly as a non-woven viscose bamboo fiber. One goal of the invention is to have a diaper with a soft top or back sheet that allows fast evaporation and wick-off of any moisture. Another goal of the invention is to provide a diaper having anti-bacterial and anti-fungal properties. Back sheet 101a is provided as a viscose bamboo fibrous non-woven material. Diaper 100 is formed of layered materials pressed together using heat. This particular view depicts the top front portion of diaper 100 (when placed on a child) to include attachment side ears 102. Attachment side ears 102 are fabricated from a non-woven biodegradable material such as viscose bamboo that may be micro-perforated to reduce moisture and to allow breath ability of the child's skin beneath the tape attachment interface. Attachment side ears 102 may be fabricated from bamboo or another organic biodegradable materials. Side ears 102 are positioned between the back sheet 101a and a breathable plant based poly film layer just under the back sheet and the front sheet 101b (not visible in this view), which is the same bamboo material as the back sheet.

Diaper 100 includes a pair (left, right) tape attachment interfaces 105 located at the lower end of diaper 100 opposite from the attachment side ears 102. Each tape attachment interface 105 includes an elongate tape strip 106 (folded over the leading edge) for fastening the diaper from the rear to the front at side ears 102. Interface 105 may be fabricated from a bamboo viscose sheet pressed in between the back sheet/breathable film layer and under sheet as described further above relative to side ears 102. Diaper 100 includes a front panel 103. Front panel 103 may be a biodegradable tape fabricated of bamboo or other biodegradable plant based materials used in this embodiment to reinforce the top front portion of the diaper. Panel 103 is rectangular in this embodiment and is pressed onto the surface of the bamboo back sheet 101a.

Diaper 100 includes opposing stretch panels 104 that may be formed in the layered materials by first bunching and then sewing or pressing (hot press) the materials together such that the layered material swath gains a linear elastic stretch capability so as to hug comfortably to the child's legs to prevent moisture or fecal materials from escaping the core absorption layer of the diaper while being worn. The core absorption layer of diaper 100 is described in detail later in this specification. A third stretch panel 107 is provided in this example and may be formed in the same way mentioned above for opposing stretch panels 104. Stretch panel 107 is designed to enable elasticity at the back waist portion of diaper 100 when worn enabling a better fit and reducing the possibility of moisture, fecal matter, and stench from a soiled diaper from escaping the absorption core materials of the diaper.

Diaper 100 includes an acidic level sensor 108, in this embodiment, provided of the form of a PH strip for measuring alkaline versus acidic material environment. PH strip 108 is embedded into the core materials of the absorption layer in this embodiment. Strip 108 changes color to indicate to an observer the presence of acidic moisture (urine) in the absorbent core materials of the diaper near the strip. Strip 108 may indicate one state of acidity versus alkalinity or in effect may detect a urine state of diaper 100. A normal urine acidic level is approximately 6.0 PH on a PH scale from 0 to 14 where 7.0 PH is a neutral PH level. In one embodiment, strip 108 may be adapted to measure more than one level of urine acidity or alkalinity. In such an embodiment a user may see indication that the diaper has been soiled, but also may gain an understanding of the current acidity/alkalinity of the child's urine, which could alert the user to a possibility that the child has a medical condition, the manifestation thereof producing an other than normal PH reading (6.0) for urine.

One with skill in the art will understand that diaper 100 may be manufactured in various sizes accepted by industry personnel as standard sizes from new born (NB), small, (S) medium (M), large (L), and extra large (XL). One with skill in the art of disposable diapers will understand that diaper 100 is a layered product with a strategically designed order of specified overlapping materials intended to provide the utility of the diaper function as a whole and to provide a more viable product for composting as well as, to reduce instances of rash, infection, allergic reaction, or skin abnormalities due to wearing the diapers, which may occur when an inferior materials selection is made. A same layered architecture including the primary material layers may be used in constructing pull-up disposables and trainer short disposables for older children without departing from the spirit and scope of the present invention.

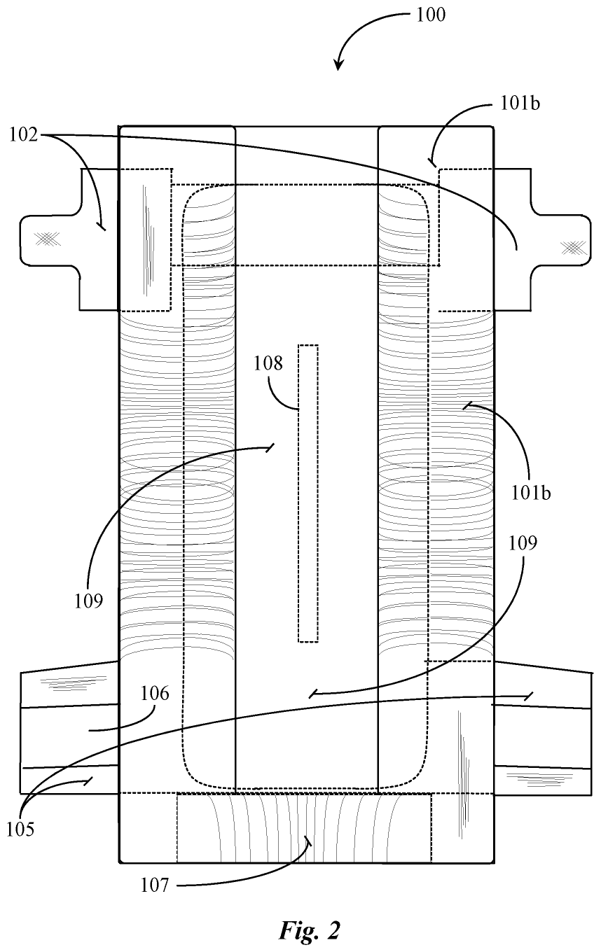

FIG. 2 is an underside view of diaper 100 of FIG. 1. Diaper 100 is flipped over left to right or right to left to depict an underside view of the surfaces that come into contact with the child. The back sheet or under sheet 101b is the same material as material 101a organically processed bamboo described succinctly as a non-woven viscose bamboo fiber. Stretch panel 107 encompasses the entire layered swath of diaper materials and provides the snug fit elasticity for fitting the diaper around the waist of the child. Similarly, stretch panels 104 encompass the entire collection of layered materials and are visible in both views.

Diaper 100 includes opposing elastic leak guards 110 extending toward center and overlapping the core absorption materials 109 on both sides. Elastic leak guards function to prevent leakage out of the core absorption materials onto the child's legs. Leak guards 110 are made of hydrophobic non-woven fabric, which may or may not include spandex, which is a rubber. Absorption core 109 includes an interfacing surface layer adapted as an acquisition and diffusion layer (ADL). The ADL layer is the inner most layer interfacing with the child and acts to more efficiently absorb and diffuse moisture into the diaper absorption core 109 that includes further components for enhancing moisture absorption.

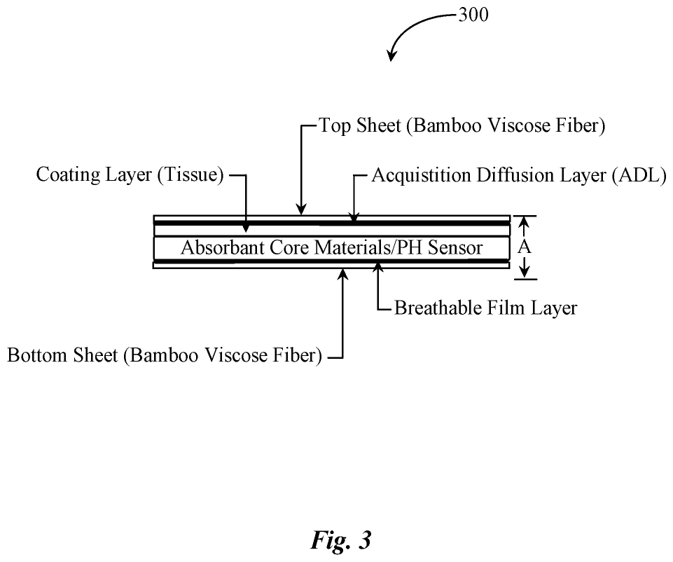

FIG. 3 is a block diagram 300 depicting the basic material layers of the diaper of FIG. 1. Block 300 represents a cross section of the diaper of FIG. 1 made through the absorption core 109. In this view, the side of the diaper interfacing with the child is facing up exhibiting a sheet made from bamboo viscose fiber analogous to materials 101b of FIG. 2. The top sheet in this chart is the interfacing sheet. The next level is the acquisition diffusion layer (ADL) that forms the interfacing surface of the absorption core 109 of diaper 100. The ADL may be manufactured of non woven bamboo materials and may possess anti-bacterial, anti-fungal, and hypoallergenic properties. The ADL layer is very soft but is hydrophobic (repels moisture) and therefore, is an ideal material for distributing moisture to and within core absorbent layer 109 while also keeping the child's skin dry.

A next layer of diaper absorption core 109 is a fast absorbing tissue layer referred to herein as a coating layer. The tissue quickly pulls moisture through the ADL and further into the core. The primary layer of the super absorbent core 109 is the absorbent core materials layer, which includes biodegradable super absorbent polymer (SAP) mixed in with chlorine free or unbleached bamboo pulp. The SAP is a starch grafted sodium polyacrylate wherein the industrial technique of starch grafting shortens the long train like structure of the compound such that microorganisms can reduce it very rapidly compared to other SAP resins. This material selection improves the compost ability of diaper 100. The absorbent core layer with PH sensor is bounded by another tissue layer to help further absorb moisture.

A next layer of protection is provided in the form of a biodegradable and breathable plant-based poly film. This layer is designed to prevent moisture and odors from escaping the tissue layer and to still allow the tissue layer to get air. A bottom sheet that is the most exterior sheet away from the child's skin is provided in the form of a non-women bamboo viscose fiber, same material as the top sheet. The biodegradable poly film layer is an agent that speeds up the composting process for all of the biodegradable materials.

Empirical composting analysis provided by SGS (quality control testing firm) in an independent manner indicates a compost state percentage of the whole product over time. For example, at day 15 under proper composting conditions, diaper 100 is already 40 percent composted. By day 30 it is over 50% composted. By day 75, the diaper is at least 60% biodegraded. The biodegradability is dependent on the selection of materials used to fabricate diaper 100 and the material location in the aggregate layering of those materials in the diaper. The accelerated biodegradability of diaper 100 lends to a greater interest in composting diapers over letting them collect in a non-oxygenated land fill where biodegradability is compromised.

One goal of the present invention is to account for the use of diaper 100 over time and enable automated notification to a composting service to pick up dirty diaper loads for direct composting. Another goal of the invention is to grant carbon offset credits to user's who compost their dirty diapers.

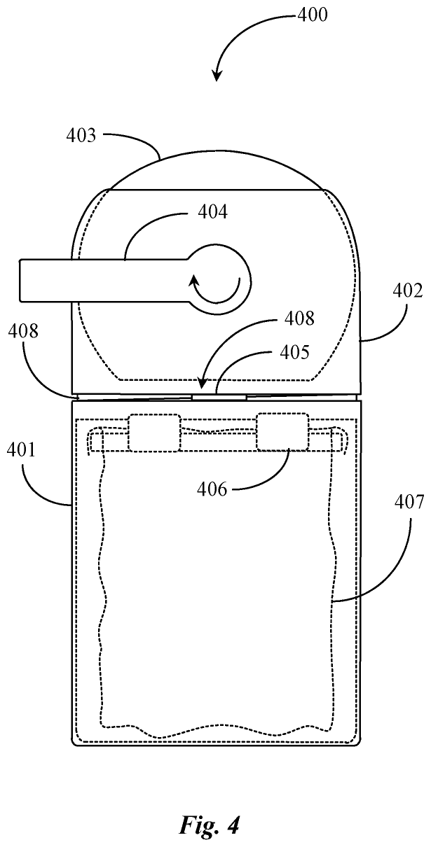

FIG. 4 is a side elevation view of a diaper pail repository 400 for containing used diapers depicted in a closed and locked position. Diaper pail 400 is a diaper repository adapted for accepting soiled diapers for later composting. Diaper pail 400 is annular in this example and may be assembled from plastic molded parts. Diaper pail 400 includes a lower body 401 having a closed bottom or floor and an open top end. Diaper pail lower body 401 contains a circular mounted bag stationing frame 408 adapted to hold a biodegradable plastic diaper disposal bag 407 via spring loaded bag clips 406 in an open position to receive soiled diapers.

Diaper pail 400 includes a plastic upper body 402 that is open at both ends but where the top end includes a domed radius and smaller opening that may be gasket sealed using an o ring type dry seal gasket that may tolerate repeated opening and closing of the opening. Upper body 402 houses a diaper transfer sphere 403 that may be rotably suspended by mounting within body 402 in a manner to be concentric with the enclosing space. Transfer sphere 403 may be molded from plastic and has one open end adapted to align with the opening in body 402 upon rotation thereof using a rotation handle 404 attached to transfer sphere 403 through the wall of upper body portion 402 of diaper pail 400. Rotation handle 404 may be a molded plastic part attached to turn axles extruding from transfer sphere 403 through the upper body 402.

In one embodiment, transfer sphere 403 may be locked into a closed position as depicted in this view with the rotation handle rotated to approximately 90 degrees and wherein the opening of the transfer sphere is facing downward toward biodegradable bag 407. A locking lever or plunger stop 405 may be provided at the bottom of upper body 402 and may interface with rotation handle 404 to lock rotation handle 104 in place sealing off the top opening of upper body 402 from the outside. A dry gasket may be placed around the inside of the domed end of the upper body portion to interface with the annular wall of transfer sphere to lock order inside the diaper pail. Stop 405 may be a lever that a user may depress, pull out, or slide over to unlock rotation handle 404 from a closed position where the open end of transfer sphere is facing straight down with the ball end sealed against a gasket. A gasket may also be placed around the inside edge of lower body 401 near the top or around the inside edge of the upper body portion 402 to seal both parts together in assembly and to prevent odor from escaping around the interfacing edges of the molded parts.

In one embodiment, additional odor-absorbent/masking materials may be incorporated into diaper pail 400 without departing from the spirit and scope of the present invention. For example, a transfer sphere material liner for transfer sphere 403 as well as a barrel material liner may be provided for lower body 401. Oder absorbent materials may also include scents that are deemed safe to use. However, organic non-scented versions of these materials may be made available such as to those parents and or children that have known allergies and need more than just the diaper to hypoallergenic.

In order to place a soiled diaper into diaper pail 400, a user manipulates stop lever or plunger 405 to unlock rotation handle 404, which then may be freely rotated 180 degrees to open the transfer sphere to accept a soiled diaper according to the direction of the arrow positioned about the mounting axis of the transfer sphere. In one embodiment, diaper pail 100 includes some electronic circuitry 409 to enable the pail to keep track of the number of times the transfer sphere 403 is physically opened to accept a soiled diaper. In this embodiment, circuitry 409 is mounted on the inside of diaper transfer sphere 403. In one embodiment a camera or other optical component may be provided for recording diaper disposal events without departing from the spirit and scope of the present invention. Circuitry 409 includes a battery, an electric counter based on the motion of the transfer sphere, a memory to store data, and at least a communications chip for communicating data results to a service node for reporting over time, the number of diapers that have been placed in diaper repository 400.

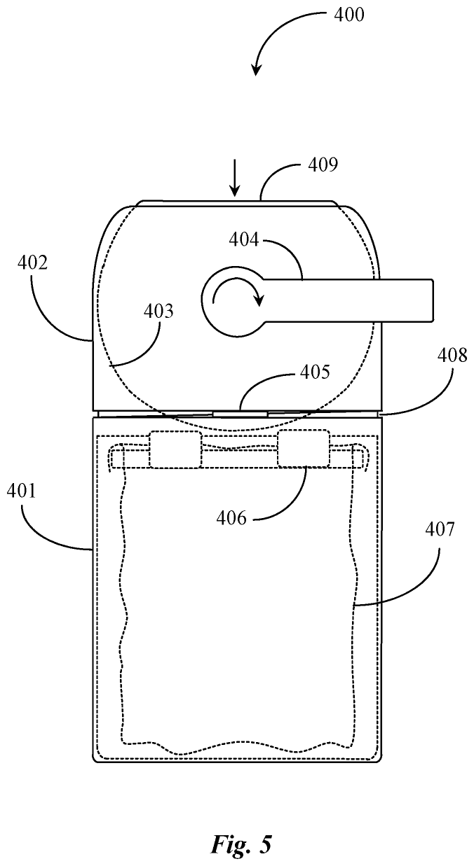

FIG. 5 is a side elevation view of diaper pail 400 of FIG. 4 depicted unlocked and opened to receive a used diaper. In this view, diaper transfer sphere 400 is rotated 180 degrees to open state placing the open end 409 of transfer sphere 403 in an upward facing position to accept soiled diapers. To open pail 400 to accept diapers, a user manipulates stop 405 to unlock rotation handle 404 and then rotates handle 404 approximately 180 degrees to align opening 409 of transfer sphere 403 with the opening atop upper body 402. The user then places the soiled diaper or diapers through the opening in transfer sphere 403. The user may then close handle 404 by rotating it back to the first closed and locked position. Stop 405 may click in automatically to lock handle 404 in place by using spring technology and the appropriate mechanical interfaces between the lock mechanism and the handle.

Upon closing rotation handle 404, the diaper or diapers in the transfer sphere drop through bag stationing ring 408 and into biodegradable plastic bag 407 through the supported bag opening. The gaskets prevent odor from escaping after the diaper pail is closed again. In one embodiment, transfer sphere 403 is enabled with a volume sensor (not illustrated) to detect temporary volume within the sphere at the moment of transfer of one or more diapers to the bag within the lower body. In this way, a user is not restricted to tossing one diaper at every disposal event to keep an accurate count of disposed diapers. A volume sensor may be a weight sensor or an optical sensor.

A counter (not illustrated) may be triggered to ad to an existing count of how many soiled diapers reside within the biodegradable bag. In a preferred embodiment, the counter implement may be reset to zero when the bag full of soiled diapers is removed from the diaper pail. In one embodiment, upper body 402 may be threaded onto lower body 401 wherein a half turn will be sufficient to remove the top apparatus to access the diaper bag. In one embodiment of the invention described in more detail later in this specification, an artificially intelligent (AI) accounting system mitigates reported diaper use figures against diaper order quantities delivered each month and uses the disposal data to fine tune order quantities to prevent unnecessary stockpiling, or running short at the user end. More detail about access and use of the diaper of the present invention is provided later in this specification.

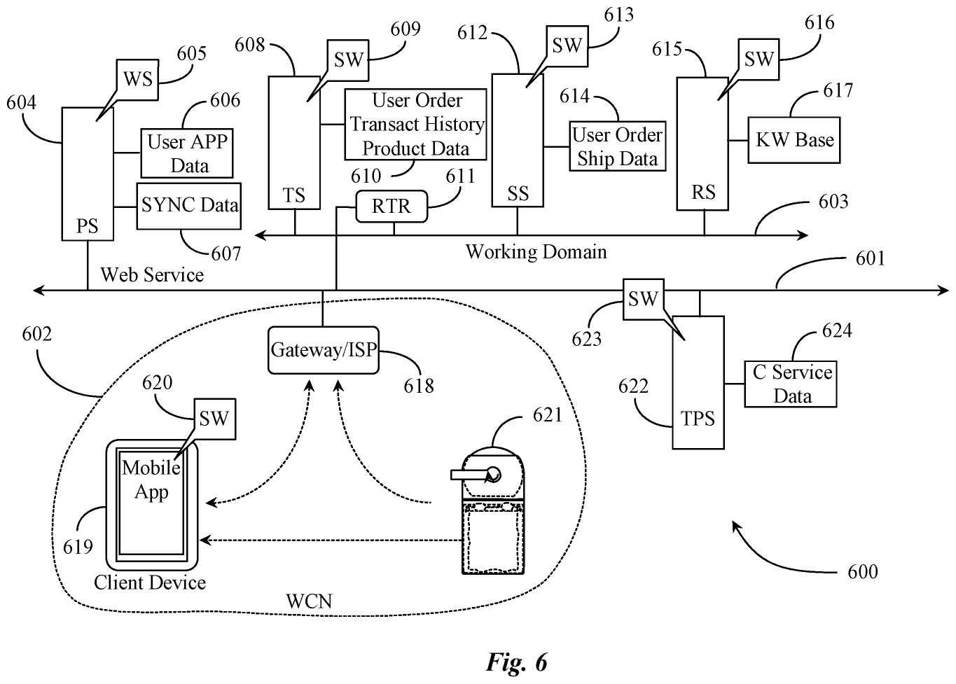

FIG. 6 is an architectural overview of a communications network 600 supporting a diaper fulfillment service according to an embodiment of the present invention. A diaper fulfillment service shall mean a service that a user may subscribe to that may deliver a strategic number of new diapers to the user each month and that may, through monitoring, fine tune the number of diapers delivered to the user each month, and that may notify and arrange for pickup of the used diapers set aside in a diaper pail analogous to diaper pail 400 of FIG. 4, wherein the picked up diapers are taken to a composting facility instead of winding up in a land fill as would occur if the user tossed soiled diapers into the public curbside refuse management system.

It is duly noted herein that the motivation to compost soiled disposable diapers is much more at the forefront amongst users given the superior compost ability of diaper 100 over current art diapers, and due to an innovative process that awards purchased carbon offset credits to users who compost and applies the credits to future diaper purchase orders saving money. Current art diapers include non-biodegradable components and biodegradable components that are slower to biodegrade and therefore are not routinely taken to a composting facility but rather disposed in a land fill. Documentation of monetary benefit by earning carbon offset credit and relative user testimony aids a marketing platform to recruit more users to sign up for organic bamboo diaper fulfillment services.

Communications network 600 includes the well known Internet network referenced herein as a network backbone 601. Network backbone 601 represents all of the lines, equipment, and access points that make up the Internet network as a whole including any connected sub-networks. Therefore there are no geographic limitations on the practice of the present invention. Network backbone 601 may also be referred to in this specification as Internet 601. Backbone 601 may be that of a corporate wide-area-network (WAN), a private WAN, a municipal area network (MAN) without departing from the spirit and scope of the present invention.

Internet 601 is accessible through a gateway 618 hosted by an Internet service provider (ISP). Gateway 618 may bridge a wireless carrier network (WCN) 602 to Internet 601 for connectivity. Internet 601 supports a page server 604 adapted to serve web pages and websites to users upon user request. Server 604 hosts a website (WS) 605. WS 605 may serve as an access point for clients to join and subscribe to the diaper fulfillment service of the invention. Server 604 has connection to a data repository 606 adapted to contain data about subscribed clients and data about a client software (SW) application 620 that may be provided for download to clients such as on a client mobile phone 619, the client wishing to join the network.

SW 620 is depicted as a mobile application on a client smart phone 619 that has access to Internet 601, server 604, and website 605, through gateway 618. SW 113 may be downloaded and installed on smart phone 109 by an operating client. In this embodiment, an operating client is a pregnant woman seeking to help herself and her baby to flourish nutritionally during pregnancy and after the baby is born. SW 620 is a dedicated thin client application that provides a vehicle for passing data to the fulfillment service for processing and a user interface for ordering diapers and other products that are specially adapted for women and their babies.

A client operating smart phone 619 aided by SW 620 may have access a diaper pail 621, which is analogous to diaper pail 400 described in FIG. 4. Diaper pail 621 may be adapted as an Internet of Things (IoT) device that may independently report diaper use statistics to a node that is responsible for fulfillment calculations for the subscribed user. Diaper pail 621 may be aided by electronic circuitry as described further above with respect to FIG. 4 components 409. Circuitry 409 might contain a SIM component for contacting a server and momentarily connection to the server over network 600 to report information. Typical information reported by pail 621 is diaper count of soiled diapers contained in the pail. In one embodiment, diaper pail 621 is enabled with Bluetooth technology and may report data to mobile phone 619 running client application 620.

In one embodiment, a client may be a user with one or more diaper aged children who also has a data tracker and application for reporting tracked data and for transacting over the network for vitamins and supplements optimized for women with young children or whom are currently pregnant with young children. The fulfillment service of the present invention may be added on to a fitness tracking and advisory service without departing from the spirit and scope of the present invention.

Diaper pail 621 is adapted with sensor to report number or volume of soiled diapers that pass through the transfer sphere of the pail. The diaper pail may indicate updated volume periodically and may report that data to another network node such as a server or an application running on a communications device. Network backbone 601 supports a service working domain referenced herein by a sub-network backbone 603 having access to Internet backbone 601 through a router (RTR) 611. Sub-network 603 may be an Ethernet network hosting, for example, a transaction server 608 aided by software (SW) 609.

Working domain 603 represents the back-end part of the fulfillment service of the invention in this embodiment. Working domain 603 may be a set of reserved cloud servers and repositories and data processing tools (SW) that are strategically adapted to provide a stable supply and distribution chain to carry out fulfillment services for the user for diapers, disposable biodegradable briefs or pull-ups and other related accessories like baby wipes, baby creams, lotions and of course the diaper pail and biodegradable diaper bags.

Working domain 603 has connection to Internet 601 through data network router 611 such as with an Ethernet network for example. Backbone 603 supports secure transaction server 608 aided by SW 609. Transaction server 608 has connection to a data repository 610 containing user secure order information, user transaction history, and updated product information including price and availability. In one embodiment, a user operating mobile phone 619 with mobile application 620 running may, through the application, connect to WS 605 and then be redirected to transaction server 608 to carry out a transaction for diapers, and or other products.

Page server 604 has a data repository connected there to and containing user data for syncing to the mobile application 620 providing information, advertising, tips and tricks for parenting, and other like supportive content for new mothers and long time mothers raising children that require diapers and other baby products. Working domain 603 may report activity and recommendations relevant to users participating in the service back to WS 605 for update of a local data set held for each user that may be synced down to the user's mobile application 620 when the user uses the application to log into WS 605 hosted on server 604.

Backbone 603 supports a data processing server 612 aided by SW 613 labeled a user order tracking server. Tracking server 612 monitors a fulfillment supply chain and tracks orders and shipping states and creates order and use statistics history that may be reported back to server 604 or accessed through server 604. SW application 613 is adapted to receive aggregated data, normalize that data for processing, and processing the received data to results that may be then maintained in a server-connected data repository 614 adapted to contain current user order status data and shipping status data, as well as, data pertaining to actual ongoing use of at least shipped diapers for each subscribing user.

Order tracking server 612 may receive data from a client such as one operating smart phone 619, for example. In one embodiment, working domain 603 is a cloud-based data service where server 604 and website 605 function as a proxy brokering the connection between cloud-based services and the client device. Client device 619 running SW 620 may access all of the transaction history, product use history, and current order status and delivery expectations data from WS 605 through the mobile client application 620, which includes at least a home page or dashboard that presents and or includes an access link to all of the updated information relevant to a subscribing user's activities and experiences with the service.

It is noted herein that a subscribing user operating smart phone 619 aided by SW 620 may practice the invention with only the client application without departing from the spirit and scope of the invention. However, use statistics and disposal statistics of shipped diapers and accounting for that history is critical for each subscriber for stream lining the process. It is also clear to one with skill in the art that the client may use all of the illustrated devices or any combination thereof without departing from the spirit and scope of the invention.

Sub-network backbone 603 supports recommendation server 615 hosting software (SW) 616. Server 615 may access data from transactions, orders, and from WS 605 in the process of determining whether to make a recommendation to a user and send that recommendation to the WS and eventually to the user's mobile phone or not.

Recommendation server 615 includes a data repository 617 adapted to contain knowledge based data relative to bio degradable materials and processes, knowledge data contained in repository 617 may be relative to diaper functions, diaper materials, diaper constructions, use methods, disposal methods, composting information and knowledge data relevant to other benefits of organic diapering and composting including carbon offset.

A recommendation to a user from server 615 aided by SW 616 may be one to increase or to decrease a monthly ship amount of diapers based on analyzing diaper use statistics. Another example of a system recommendation to a user might be one to recommend a larger size diaper based on data about the child that has been monitored and analyzed for recommendation or no.

SW 612 outputs the resulting processed data to recommendation server 615 running recommendation SW 616. Recommendation SW 616 is adapted to read results received and determine, with additional calculation if necessary, whether any recommendations or special insights or other notifications may be ordered for the user because of a noted shift or deviation of certain data from a base norm data value. Recommendation server 615 aided by SW 616 may create general notifications for encouragement, congratulations on goal achievements, and tips or advice relative to any product data.

In one embodiment, SW 616 may participate in chat conversations with a user/subscriber through mobile phone 619 and running application 620 using artificial intelligence (AI), optical recognition for images and text, bar code scanning (OCR), and interactive voice recognition capabilities (IVR). Data within repository 617 may include results and methods of clinical trials and knowledge data resulting of clinical study along with general knowledge data surrounding diapering where it applies to biodegradable disposable diapers. A user may maintain a subscription as long as diapers and related products are needed.

In one embodiment of the invention the parent has given the child's data statistics including weight, length, etc. and the service recommends a diaper size and quantity to be shipped to the user. However, additional information may be provided by the parent to the service such as disclosing the child's daily diet, for example. This may be accomplished through the parent mobile application using OCR scan to scan in a bar code of the baby formula or food product fed to the child. Repository 617 may include researched information relative to early digestion and certain foods that may be more or less digestible relative to time.

Accumulation of diet data may be used to predict likelihoods or to create weighting factors for one or more predictive algorithms, in one case, an algorithm that predicts a higher or a lower diaper requirement in terms of monthly amount. On the flip side of the embodiment, the monitored use of diapers by a child may be used to validate one diet over another diet where digestibility is concerned. For example, a notification sent to a parent may recommend a food, liquid formula, or diet or supplement that is healthy and results in less frequent defecation and or urination for the child. A goal of AI is to be enabled to fine tune the amount of diapers, for example, shipped each period to match actual use statistics. By comparing data over larger geographic service areas, additional knowledge may be discovered that may be useful for parents and children in a specific region to make adjustments in diet or other changes like adjustments in sleep periods.

Backbone 603 also includes a third party server 622 aided by SW 623. Third party server 622 may be hosted by a composting business or non-profit that may be notified when local residents within the sphere of service have soiled diapers for compost. SW 623 may process notifications relative to the introduction of new fulfillment service subscribers as composting customers and the current need for them to have diapers picked up for compost. Server 622 has connection to a data repository 624 adapted to contain composting service data and user data for the users that use the business to retrieve and then compost the soiled diapers.

In one embodiment a user may determine to do his or her own diaper composting without relying on a pickup service for that. In another embodiment, some users that are subscribing to organic bamboo diapers through the service of the invention may elect to continue to use the land fill as a destination for the soiled diapers. However, incentive may be made to users by purchasing carbon credits and then giving users lower monthly prices on diapers if they agree to allow composting of the soiled diapers and that composting is performed by professional compost business entities. In one embodiment, a user has access through mobile application 620 to an emergency order interface or link button that may be executed through the application while logged into WS 605 to register an emergency diaper order for quick ship in situations where a user runs out before a next shipment is due, or a user is traveling or away from the primary ship address and has no diapers but needs them to be delivered to a temporary address site where the user might be staying temporarily or vacationing, etc.

In general use, a user operating client device 619 may, after downloading and installing mobile application 620, navigate to WS 605, log-in, and place a monthly subscription order for the main product, which are the disposable diapers. The order may include other secondary products which may also be included in the purchase order. The user may also agree to compost to take advantage of the price reduction afforded by the purchase and redistribution of carbon credits to composting users. Once an order is active, the user may monitor the progress of the order and may modify it. The AI system may count how many diapers a user has gone through in a month, for example, and then may determine whether to recommend increasing or decreasing the quantity of diapers shipped the following month. Over time the system learns to be efficient in that it keeps up with the actual diaper use through the diaper pail device 621.

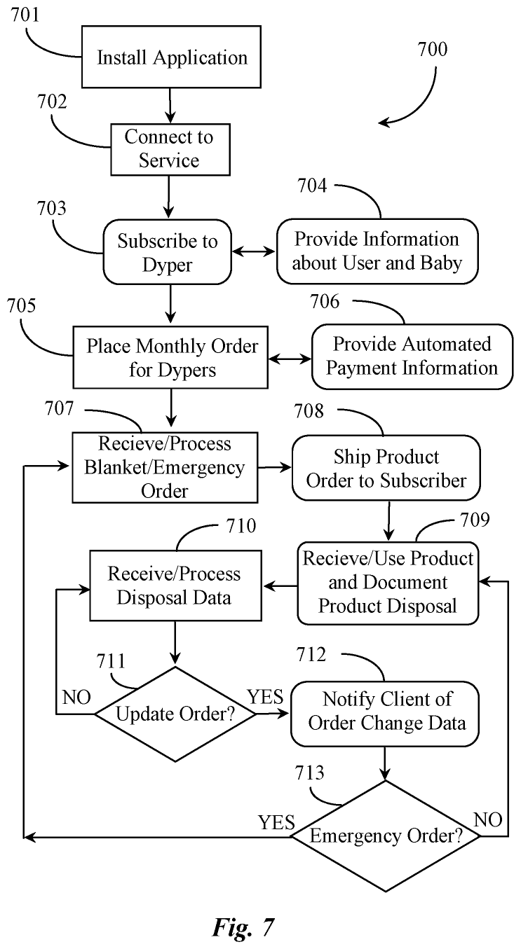

FIG. 7 is a process flow chart 700 depicting process steps for starting and managing a diaper order according to an embodiment of the present invention. In a preferred embodiment, a user is a subscriber to a monthly diaper delivery service that delivers a negotiated or recommended amount of diapers to the user. A goal of the service is to deliver the number of diapers that are expected to be used in the period of time covered. The system includes an AI accounting and reporting method to document the actual use of the shipped diapers during the period (Month).

A user and potential subscriber may visit the Web site of the service and download a thin client SW application analogous to SW 620 on mobile phone 619. At step 701 the user may install the application. At step 702, the user may through the mobile application, connect to the fulfillment service. At step 703, the user may request a subscription to the diaper service trade marked as Dyper™. At step 704, the user may be asked to provide information about themselves and the child or children for which diapers will be purchased and delivered. The interaction may include registration, and form filling or textural interaction through the mobile application. In this overall process, the user may look at products and browse information about those products.

At step 705, the user may initiate and place a monthly order for diapers that includes an amount to ship each month and automated debit of a payment account provided by the user, or in one embodiment, created for the user. If an account is created for a user, then the user may fund the account prior to shipment of diapers. The user may provide automated account payment information to the service through the mobile application at step 706. In this aspect, the user is placing an order at the Website which then places the order with an appropriate entity in the service supply chain that includes the delivery address of the user. For example, in one aspect, the diapers are warehoused in a number of houses that are regionally distributed to provide quick and efficient delivery of the product to the ordering user.

At step 707, a warehouse location receives the placed order from the Website and processes the order for shipment. In typical ongoing business the order is an ongoing purchase order authorizing monthly shipment of an agreed upon number of diapers to the ordering user. In one embodiment an order may be an emergency order for diapers initiated by a user through the mobile application by selecting a prepared option for starting an emergency one time shipment. In one aspect, an emergency shipment is free of charge but must be limited in scope and frequency. For example, if a user gets isolated somewhere for a period without access to diapers at home for whatever reason, an emergency order might be placed to direct a free shipment of diapers to an address provided by that user placing the emergency order.

At step 708, the distribution warehouse that received the order ships the diaper product monthly order to the user address. In step 708, the warehouse may use one of a variety of available shipping means such as UPS, Fed EX, US postal mail, robot delivery, Drone delivery, or other accepted forms of delivery. It is a goal of the fulfillment service to deliver diapers very quickly in an emergency such as within hours of receiving the order. Monthly orders are more regular and planned shipping may be set by default where the shipments occur automatically once per month or in some cases twice per month. Once an order is processed it is also documented and added to a user transaction/order history such as in repository 610 connected to transaction server 608 aided by SW 609. At step 709, the user receives the diapers and begins to user the product(s). Diapers may arrive in conjunction with ordered accessories like baby wipes, baby creams, or similar products. The frequency of reorder of these products may be based in part on the expected use number compared to number of diapers ordered. Also at step 709, a user disposes of the soiled diapers into a Diaper pail bin

As a user traverses diaper technology the user may decide to help document diaper disposal for compost. A user may acquire a smart diaper pail, analogous to diaper pail 400 of FIG. 4 or diaper pail 621 of FIG. 6., to place all of the used diapers into for storage until full. Biodegradable diaper pail bags (not illustrated) may be used to house the diapers within the pail and the user may clip or tie up the diapers in their biodegradable bags and store those for eventual pickup for composting. The diaper pail includes a wireless communications capability for connecting to a network and reporting data recorded on the device by uploading the recorded data to a server. In step 710, the server receives and processes volume data relative to the amount of dirty diapers the user has put into the pail.

As a user logs dirty diapers through the diaper pail, the service may make a determination after receiving and processing volume data, whether the service will recommend a change in monthly order amount at step 711. For example, if the diaper pail count is ahead of expected use for a time period such as two weeks or behind the expected use for the period then step 711 may be a determination made by the system. At step 711, if the system does not determine to recommend a change order to increase or to decrease shipment amounts based on disclosed actual use data, the process may loop back to step 710 where receipt of actual use volume data is sent, received, and processed.

If the system determines to recommend a change order at step 711, the process continues to step 712 where a recommendation may be generated and a notification may be sent to the user, the recommendation suggesting an increase in product amount to offset higher use figures or a decrease in product amount to offset lower use figures and prevent over stocking at the user's residence. The calculations may be performed by SW 613 on server 612 and or by SW 616 running on server 615. In either case a recommendation server 615 may be dedicated to getting notifications out to user accounts in a timely manner. In one aspect, a result and notification may prompt a user to consider whether or not to initiate an emergency order of diapers at step 713. An emergency order can be initiated by the user at any point by selecting an SOS link to the transaction server 608 aided by SW 609, the link initiating an emergency order transaction where payment may be waived for the extra amount of diapers ordered. There may be some limits placed on the number of times an emergency order might be transacted over a period of time. An emergency order may be placed for different reasons, for example, the user has used more diapers than can be replaced under the current monthly order amount shipped, or the user has the diapers but has traveled somewhere and forgot to bring them or lost them in luggage, etc. The corrective change order may be recommended to correct the shipping number to prevent another shortage or to stop an overflow of diapers shipped but not being used in the month. If at step 713, the user initiates an emergency order, the process may loop back to step 707 to process the emergency order. If at step 713, the user determines not to initiate an emergency order, the process may loop back to step 709 where normal product use and documented disposal ensues.

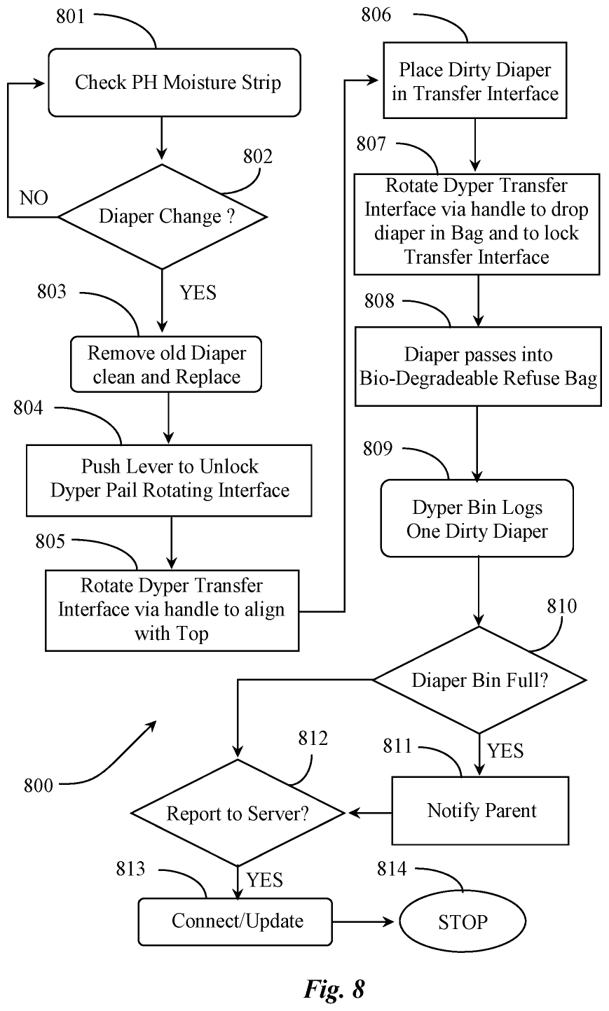

FIG. 8 is a process flow chart 800 depicting process steps for operating a diaper repository to accept diapers for disposal and updating use statistics according to an embodiment of the present invention. The diaper of the present invention includes a PH strip to test for presence of urine acidity in the core of the diaper. At step 801, a user may check the PH strip on the diaper being worn to determine if the diaper should be changed out for a new one. At step 802, the user determines if the PH strip indicates a diaper change. If at step 802, the user determines the PH strip has not indicated a diaper change is necessary, the process loops back.

If at step 802, the PH strip indicates a diaper change should be initiated, the user may remove the old diaper, clean up the child, and replace the diaper with a new diaper. At step 804, the user may approach the diaper pail and push pull or slide the lock lever to unlock the rotating handle of the diaper pail that controls the position of the diaper transfer sphere. At step 805, the user may rotate the handle to align the opening of the diaper transfer sphere to the top opening of the upper body detachable portion of the pail. In this position, the user may dispose of one or more diapers into the diaper transfer sphere (interface). At step 807, the user may return the rotation handle back to the original closed position to lock. Locking may be automated once the handle is back in a correct position. This action causes the diaper load in the transfer sphere to drop through the diaper bag station ring mounted near the top of the lower body portion of the pail and into a biodegradable diaper bag. In the locked position, the radiused surface of the transfer sphere sets against a gasket preventing leakage of odor.

At step 809, the diaper pail logs one disposed diaper or two or more if a volume sensor is employed in place of or in addition to a simple mechanical counter that only counts the times the handle is rotated. At step 810, the determination may be made at a certain count that the diaper pail is full and needs to be emptied. If at step 810, the determination is not made that the diaper pail is full, then the process loops back until a count synonymous with a full diaper pail is reached. If at step 810, it is determined that the diaper pail is full, the system may notify the parent at step 811 that the pail is full based on latest count. The process may stop or reset at step 812. The user may replace the full refuse bag full of used diapers with a fresh bag and the refuse bag may be tied off and placed with other bags for pickup by a local composting service that may be notified by the fulfillment service when total diaper pail count reaches an amount suitable to warrant a pickup event.

In one aspect the tracking server 612 aided by SW 613 that tracks order data also tracks diaper use and disposal data and may refer that data to the recommendation server 615 aided by SW 616 to initiate an analysis and determination of notifying the user. In this aspect the notification appears in the mobile client application 620 running on the user's mobile phone 619. In another aspect, the diaper pail analogous to pail 400 of FIG. 4 may automatically notify the user via wireless protocol that the pail is full and needs to be emptied. The diaper pail may also connect and notify the tracking server. In one aspect, the service may send an order to the local compost company to schedule a pickup once an agreed number of refuse bags full of soiled diapers is available. A compost bin may be made available at curbside to accept the biodegradable bags of soiled diapers. In one aspect, users who compost their used diapers as opposed to placing them in a landfill may obtain a carbon credit that may be applied as a carbon discount on the diaper subscription.

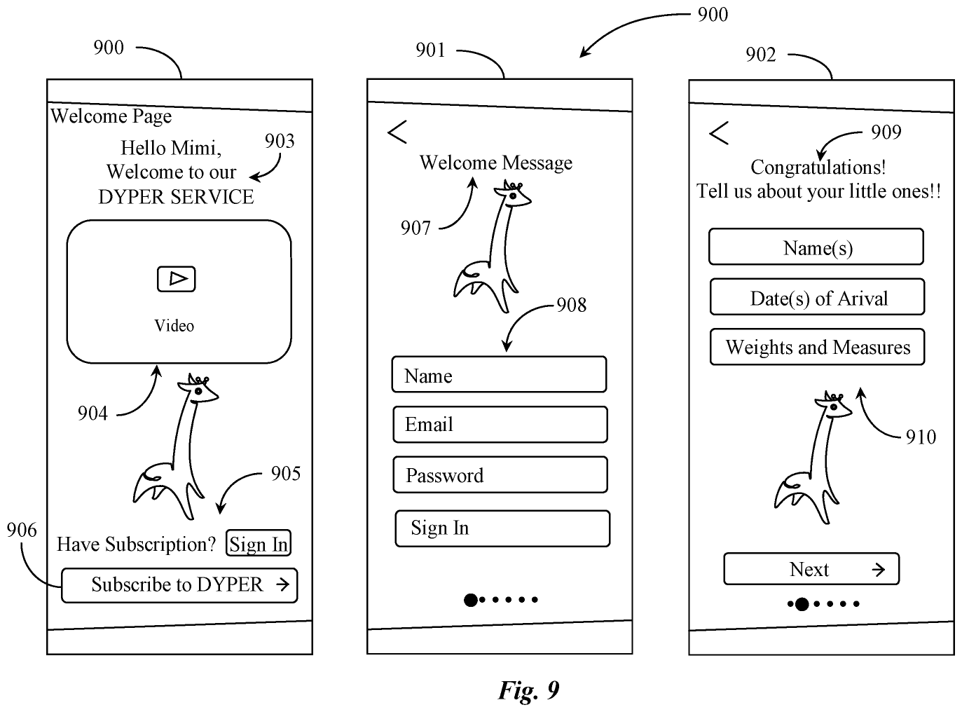

FIG. 9 is an elevation view of exemplary screen shots 900 of the mobile application 620 of FIG. 6. The mobile application 620 (see FIG. 6) is a browser-capable standalone SW application that may display a sync page or home page where a user may see all of the latest information and activity data tied to the user account. In one aspect a user must log into the Web site in order to obtain sync functionality or to receive updates and place orders.

Screen shots 900 include a welcome page 901 that a user might see when first using the application. Welcome page 901 may include a welcome statement 904 that welcomes the user to the “dyper” service. In one embodiment page 901 may include one or more information and or testimonial videos 905 allowing the user to learn about bamboo diaper fulfillment services. The welcome page may include an interface 906 for signing into the user account if the user already has an account. If not, the welcome page 901 may include a subscription link 907 to subscribe to the fulfillment service if the user has yet to obtain an account.

A user may have an account and simply log-into that account through the application and then sync with the WS data server to download the latest activity data and information about service updates, new products offered, etc. A user may also jump to the subscription process by hitting link 907. Screen shots 900 include a site authorization page 902 that may include a short welcome message 908. Page 902 includes a collection of dialog boxes 909 for the user to provide name, email address, and to create a password to enter the site and user account data.

The user may sign in immediately after starting an account and may log in using the email and password information. Once logged into the account, the user may be served page 903 to obtain some data about the diaper wearers. Page 903 may include a congratulations message 910 and a set of dialog boxes 911 requiring user input. Dialog boxes 911 ask for input about the diaper wearer such as name or names, the dates of arrival (day born), and the weight and measurements of the baby or child. Weights and measures refer to child weight and measurements like height and girth of the child.

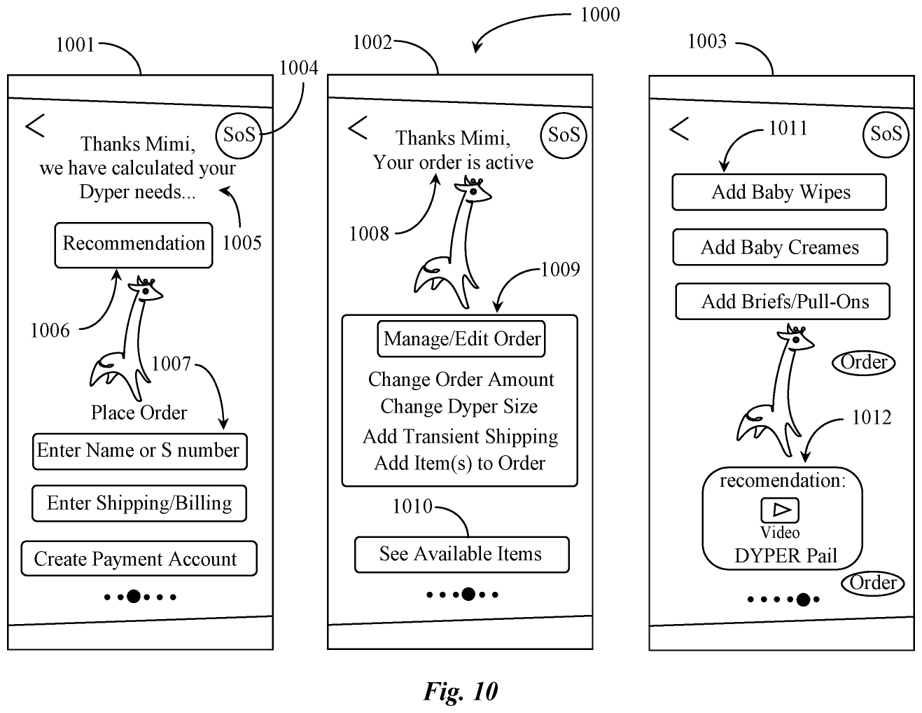

FIG. 10 is an elevation view of exemplary screen shots 1000 of mobile application 620 of FIG. 6. Screen shots 1000 include pages 1001 through 1003 relative to transacting and managing a subscription-based order. Screen shots 1000 include a page 1001. Page 1001 is a follow-up page confirming the user as a subscriber and informing the user in a top instruction 1005 of a calculation that has been made to determine the user's diaper needs. An SOS icon 1004 is provided at top-right for a user to invoke should they need to place an emergency diaper order if required.

In one aspect, page 1001 includes a list of one or more recommendations 1006, which a user may expand upon by clicking on those links. Page 1001 includes an order interface 1007 for placing a diaper order that includes a dialog box for entering name or stock number of the diapers. Interface 1007 includes a second dialog box for entering in shipping and billing information. Interface 1007 includes a third and final dialog for enabling a user to create a payment account that the service may draw on to pay the monthly subscription fee on behalf of the subscribing user.

Screen shots 1000 include a page 1002 that confirms Mimi's placed order with a thank you message 1008; Page 1002 may include an order change interface 1009. Order change interface 1009 may include a link enabling the user to manage and or edit an order that is already in place. Tasks that are enabled may include changing the product amount to ship each month, changing diaper size or type, adding a transient or temporary shipping address that takes priority over the standard shipping address, and adding additional items to the blanket order. Page 1002 may include an active hyperlink to a page where the user may see and browse available items.

Page 1003 may appear if the user elects to interact with the change order interface 1009. Page 1003 may display common order additions that a user may select and then edit to add to an existing purchase order. The user may submit the edited data to the service by selecting order, which sends the data to a transaction server analogous to transaction server 608 aided by SW 609. It is important to state herein that SW 605 [web service], SW 609 [transaction service], SW 613 [tracking service], SW 616 [recommendation service], and SW 623 [composting service] may cooperate across instances and over remote server to server connections through application program interfacing (API).

In another embodiment, the SW may be incorporated on one server as a single instance of SW having the mentioned functionality. Page 1003 may also be used to present advertising media for review to users such as a video review and recommendation 1012 advertising the diaper pail and composting service including the carbon credit incentive that might be granted in the first order. Advertisement 1012 may be associated with an order option enabling the user who does not already own the diaper pail to order it separately along with signing up for composting services and the carbon credit discount.

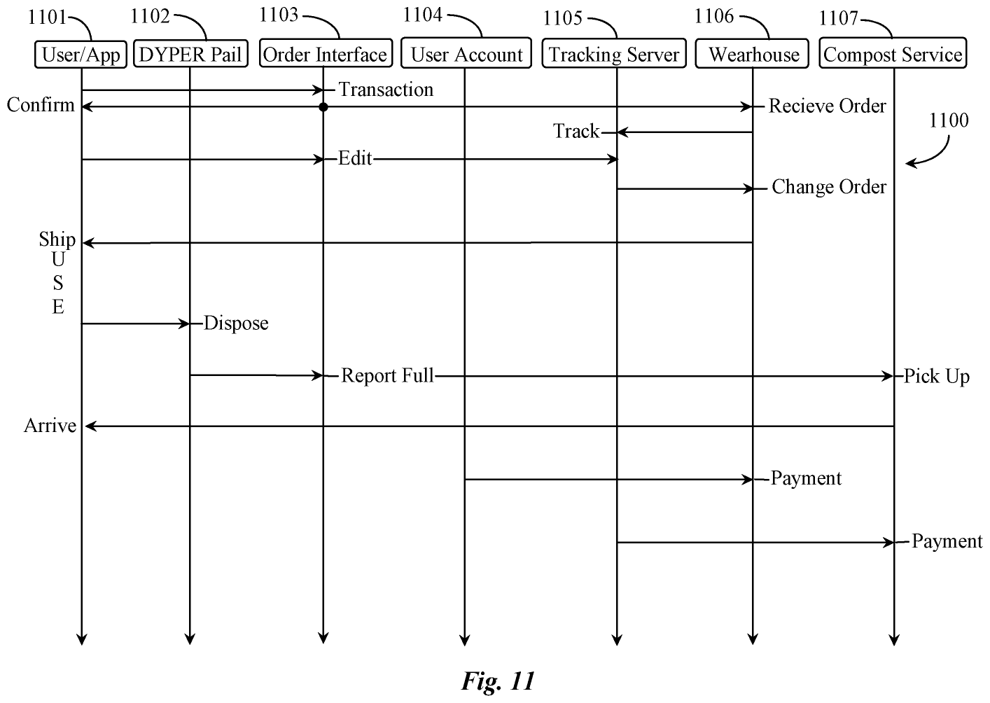

FIG. 11 is a sequence diagram 1100 depicting interaction between network nodes engaged in the service of the present invention. In one embodiment, a user/app 1101 representing a user operating a mobile phone analogous to phone 619 running the client application 620 connects to the website, authenticates, syncs data, and then initiate a transaction with a transaction server 1103 analogous to server 608 of FIG. 6 hosting an order interface supported by SW 609. Transaction server may send confirmation of order back to Website 605 and mobile node 1101. Transaction server relays the placed order to a warehouse 1106, representing any local warehouse close enough to the user to ship product to the user address in a timely manner.

Warehouse 1106 receives the order and notifies the tracking server to track the order progress and state User/App 1101 may decide to edit the existing order. The user may visit the website, sync data, and edit, at the order interface. Then the transaction server may relay that change order to the warehouse. The order items are generally kept in sufficient stock amounts to fulfill orders in a timely manner without running out of product. The warehouse 1106 ships the diapers and any accessories ordered directly to the user primary ship address.

The user then begins use of the product and begins disposing of the soiled diapers into the diaper pail 1102, analogous to diaper pail 400 of FIG. 4. In one embodiment, diaper pail 1102 may connect and report count data when it is full [refuse bag] providing the number count of used diapers in the bag. At some point order interface 1103 logs and reports the used diaper count to the other nodes and to a compost service 1107, which may arrange to pick up the used diapers at the user residence [arrive] for composting.

It is noted herein that there may be volume requirements such as a user must have three or more full diaper bags. In this case, the user account 1104 representing a user payment account electronically pays the warehouse 1106 and the compost service. In one embodiment, the user may have his or own composting capabilities and therefore may save more money by personally composting for self in place of paying a service to do that on the user's behalf. In one embodiment, one full diaper pail bag is sufficient to warrant a curbside pickup by composting service 1107. In another embodiment, a composting service makes weekly rounds picking up soiled diapers for composting and pick up whatever is left. If no soiled diapers are at the curb the composting service may simply pass by en route.

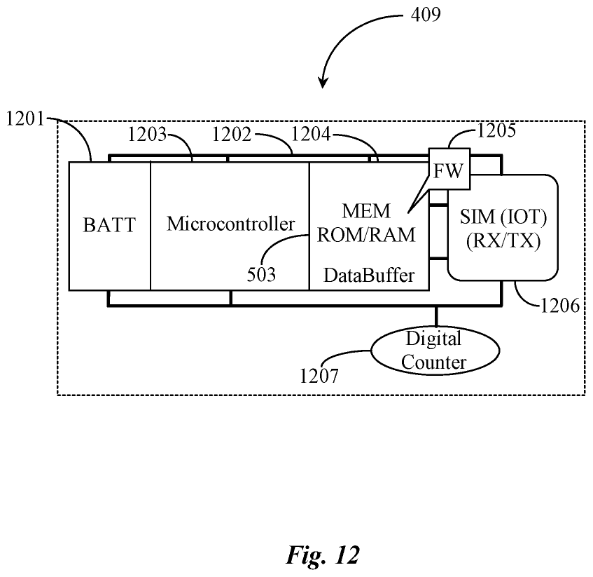

FIG. 12 is a block diagram depicting electronics components 409 of the diaper pail of FIG. 4 according to one embodiment of the invention. Components 409 include a battery (BATT) 1201. Battery 1201 may be a rechargeable battery or a set of rechargeable batteries. A micro controller 1203 is provided to boot up the unit and to control unit function and communications tasks. Components 409 include a memory block 1204. Memory block 1204 may include read only memory (ROM) and random access memory (RAM and a data buffer to stage data for upload to another device. A firmware (FW) 1205 is provided on MEM block 1204, which may be executable instruction for enabling counting and count recording of the number of soiled diapers residing in the pail.

Components 409 may include a communications chip or card 1206 enabling transmission of data and receipt of data remotely. Components 409 are connected together by a power and communication bus 1202. In one embodiment, components 409 include a digital counting device 1207. Counter 1207 is adapted to log one count every time the diaper pail handle is rotated to align the transfer sphere opening for accepting a soiled diaper. In another embodiment, the diaper pail uses a volume sensor 1208 in the transfer sphere to detect the volume of soiled diapers in the sphere. Volume sensor 1208 may be an optical sensor calibrated to detect mass and calculate volume or count, for example two soiled diapers placed in the transfer sphere instead of a single diaper.

In still a further variation of this embodiment, volume sensor 1208 is a weight sensor inside the transfer sphere that may calculate and log the temporary weight of the soiled diapers placed in the transfer sphere. In one aspect, both types of sensors are employed so a user may toss only single diapers or multiple diapers in the diaper pail, for example, if two or more children are changed together. In a preferred embodiment, diaper pail 400 has components 409 and may record data and report data independent of the user. In that case when the diaper pail is full of soiled diapers, the diaper pail may initiate a transaction with a server via connection to the server and then uploading the count data and the volume data.

In one embodiment, diaper pail 400 uses a motion sensor to boot whenever a user approaches the unit with soiled diapers, or to empty out the unit. In an embodiment with just one diaper wearing child, the diaper pail components 409 may be reduced to a resettable mechanical counter that is integrated with the rotation handle of the unit and counts the instances of handle rotation (open, close) thus calculating one diaper per open and close of the handle. In a mechanical embodiment where the diaper pail has no electronics, the user may be responsible for reporting the physical count of soiled diapers to the server. In one embodiment, where the diaper pail has communication capability with another device, the user may poll the diaper pail over Bluetooth™ or another wireless protocol to obtain the current count of soiled diapers.

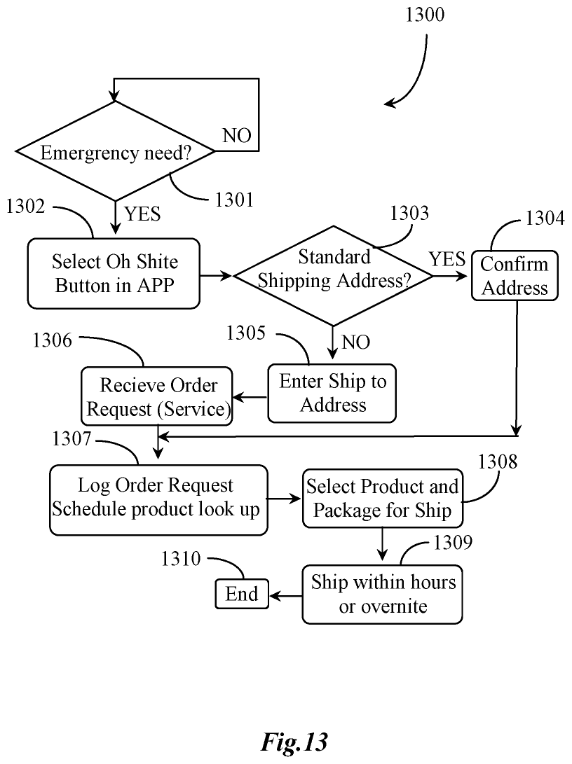

FIG. 13 is a process flow chart 1300 depicting steps for instating and fulfilling an emergency shipment of diapers. At step 1301, a user subscribed to the diaper service makes a determination whether an emergency diaper order is required. If there is no emergency at step 1301, the process loops back to normal diaper use and diaper disposal logging by the diaper pail, which is a continual process. If at step 1301, the user determines an emergency order must be placed for diapers. The user may select an SOS order button that is an integral part of the mobile client application downloaded on the user's mobile telephone. Selection of the SOS icon while connected over the network to the website may cause a redirect to the transaction server, where the order may be submitted for processing. At the transaction server, the server may prompt if the emergency order is to be shipped to the same address as the monthly shipments and the user may make that determination at step 1303.

If the user determines it is the same shipping address as the priority address then at step 1304 the system may prompt the user to confirm the address by typing yes or checking a box. The process enables the user to enter a new shipping address or select an alternative saved shipping address that will be used only for the instant emergency order of diapers at step 1305. At step 1306 the service receives the emergency order request; the request may indicate the required number of diapers the user is requesting. In one embodiment, the service may include emergency packages that contain a set number of diapers such as an estimated weeks worth of diapers.

At step 1307, the service may log and queue the user's order request and may perform a lookup of the product schedule. The order is delivered over the network to a warehouse serving the general locality of the ordering user. Product and packaging may be selected at the warehouse in step 1308 in one embodiment. In one embodiment, there are emergency diaper packs in stock for shipping. At step 1309, the distribution warehouse ships the emergency order of diapers within an hour or two of receipt of the order. The process may end at step 1310. It may depend upon the nature of the diaper emergency whether the service will recommend an adjustment in monthly diaper volume.

It will be apparent to one with skill in the art that the fast composting organic diaper and the diaper fulfillment system of the invention may be provided using some or all of the mentioned features and components without departing from the spirit and scope of the present invention. It will also be apparent to the skilled artisan that the embodiments described above are specific examples of a single broader invention that may have greater scope than any of the singular descriptions taught. There may be many alterations made in the descriptions without departing from the spirit and scope of the present invention. The spirit and scope of the present invention should be limited only by the claims provided below.

1. A disposable diaper comprising:
a first sheet of viscose fiber material disposed against a child's skin;
an acquisition and diffusion layer (ADL) interposed over top of the first sheet of viscose fiber;
a coating layer of tissue fiber interposed over top of the ADL layer;
an absorbency core layer interposed over top of the coating layer;
a permeable layer of polymer film interposed over top of the absorbency core layer; and
a second sheet of viscose fiber material overlaid over top of the permeable polymer film layer, the second sheet functioning as the exterior sheet of the disposable diaper.

2. The disposable diaper of claim 1, wherein the absorbency core layer contains a PH strip that changes color to indicate level of acidity within the core layer, the color indicated visible to a user and matchable to a chart listing PH levels associated with the colors.

3. The disposable diaper of claim 1, wherein the first and second sheets of viscose fiber are compostable bamboo fiber.

4. The disposable diaper of claim 2, wherein the absorbency core layer further includes a super absorbent polymer (SAP) derived from biodegradable plant cellulose and a chlorine free compostable wood pulp material.

5. The disposable diaper of claim 1, wherein the SAP is created by inverse suspension polymerization to avoid chemicals used in emulsification processes.

6. The disposable diaper of claim 1, wherein the first and second sheets are micro perforated to enforce dryness against the child's skin and at the external side of the diaper.

7. The disposable diaper of claim 1, wherein as a direct result of selective material incorporation and selective material exclusion, the disposable diaper is biodegradable and compostable.

8. The disposable diaper of claim 1, wherein the permeable layer of poly film is biodegradable plant based polymer that contains enzymes that enhance biodegrading efficiency of the disposable diaper.

9. A network-hosted order and fulfillment service for biodegradable diapers comprising:
a first non-transitory medium coupled to a first network server having connection to the network, the first server coupled to a data repository the non-transitory medium containing thereon instruction causing the server to:
(a) provide for download and install a network browser-based application to a user's mobile communications device having network connect capability;
(b) via the application as a user interface, receive a request from the user for a subscription to the fulfillment service;
(c) via the application as a user interface, establish a service account for a user upon request;
(d) via the application as a user interface, collect all of the data required to redirect the order request to a second network server functioning as a secure transaction server; and
(e) via the application as a user interface, sync data between the first server and the mobile communications device upon established connection, the data synced displayable through at least one electronic page of the browser-based application on the user's mobile device.

10. The network-hosted order and fulfillment service for biodegradable diapers further comprising:
a second non-transitory medium coupled to a third network server having connection to the network, the third server coupled to a data repository and having network connection to the first server, and to the second server, the second non-transitory medium containing thereon instruction causing the third server to:
(a) receive a notification to track a job order from a warehouse distribution hub;
(b) assign the job order to active tracking state and confirm the active tracking state including sending any resulting underlying tracked data back to the first network server;
(c) receive one or more activity updates from the distribution hub regarding at least one updated status of the tracked order;
(d) forward the data from step (c), to the first server for storage into the data repository coupled to the first server;
(e) receive from the first server or from a tracking device having network connect and report capability use statistics relative to diaper use and diaper disposal;
(f) pass the data to a fourth server coupled to a data repository and having connection to the third server, the second server, and the first server, a third non-transitory medium coupled to the fourth server containing thereon instruction causing the fourth server to:
(g) analyze the received data of step (f) and compare the data results with current and or future shipment numbers expected each delivery;
(h) make a determination whether or not to change order volume of the tracked order with respect to adjusting a shipping quantity to align more closely with the analyzed disposal statistics.

11. The network-hosted order and fulfillment service for biodegradable diapers of claim 10, wherein the fourth server makes recommendations based on analysis of data received from the third server.

12. The network-hosted order and fulfillment service for biodegradable diapers of claim 11, wherein the third and fourth servers are dedicated server applications hosted on a single network-connected machine.
